(19) United States
(12) Patent Application Publication
(10) Pub. No.: US 2004/0241055 A1
(43) Pub. Date: Dec. 2, 2004

(54) COMBINATION CANDLE AND INCENSE HOLDER

(76) Inventor: Mark Padilla, Santa Ana, CA (US)

Correspondence Address: LAW OFFICE OF MICHAEL P. EDDY, #510, 2683 VIA DE LA VALLE, SUITE G, DEL MAR, CA, 92014, US

(21) Appl. No.: 10/709,758

(22) Filed: May 26, 2004

Related U.S. Application Data
(60) Provisional application No. 60/473,598, filed on May 27, 2003.
(60) Provisional application No. 60/521,577, filed on May 26, 2004.

(51) Int. Cl. A61L 9/03

(52) U.S. Cl. 422/126; 431/289

(57) ABSTRACT

This invention is directed to an improved combination incense and candle holder that can be easily transported and which can use refillable fragrance solids and liquids that are ignitable. One embodiment is directed to a holder consisting of a vertically disposed base having an inner surface that is configured to be capable of accommodating a candle from the top portion of said base and that is configured to be capable of accommodating a receptacle through an aperture in said base, and a removable receptacle disposed in said aperture on a wall of said base that is capable of receiving an ignitable fragrant solid or an ignitable fragrant oil. Another embodiment is directed to a holder that is further comprised of a receptacle mounted on the top portion of the housing that is capable of receiving an ignitable fragrant solid or oil. Another embodiment is directed to a holder with a base that is further comprised of an air well disposed in said base for passage and escape of air from said receptacle. Another embodiment is directed to a holder with a receptacle with a bowl, square, star, or cylinder shape. Another embodiment is directed to a holder with a receptacle holding a stick or cone shaped incense product. Another embodiment is directed to a holder wherein the receptacle is integral to the base. Yet another embodiment is directed to a holder with a handle disposed on the receptacle.

### CROSS REFERENCE To RELATED APPLICATIONS

This application claims the benefit of U.S. Provisional Application Ser. No. 60/473,598 Filed May 26, 2003 and of U.S. Provisional Application Ser. No. 60/521,577 Filed May 26, 2004, both of which are incorporated herein by reference in their entireties.

### BACKGROUND OF INVENTION

This invention relates to candles and candle holders and more particularly to candle holders that also serve as holders for incense. Individually, candle holders and incense holders are well known in the art in various sizes and shapes. The fact that certain combustible materials can be ignited and will then disperse an active ingredient as they continue to smolder is well known. Incense sticks are examples of products made from such materials. A variety of known fragrances, for example those typically incorporated in incense sticks, can be used to release aromatic ingredients into the surrounding air and are well known to those skilled in the art. Candles similarly provide a source of fragrance as well as a source of light.

There is a need for an improved combination candle and incense holder that can be easily transported and which can be used with both disposable candles and incense sticks or cones as a source of fragrance.

### SUMMARY OF INVENTION

The present method and apparatus relate to candle holder and incense holders, in particular, combination candle and incense holders that can be easily transported and which can use refillable fragrance solids and liquids. One embodiment is directed to a holder consisting of a vertically disposed base having an inner surface that is configured to be capable of accommodating a candle from the top portion of said base and that is configured to be capable of accommodating a receptacle through an aperture in said base, and a removable receptacle disposed in said aperture on a wall of said base that is capable of receiving an ignitable fragrant solid or an ignitable fragrant oil. Another embodiment is directed to a holder that is further comprised of a receptacle mounted on the top portion of the housing that is capable of receiving an ignitable fragrant solid or oil.

Another embodiment is directed to a holder with a base that is further comprised of an air well disposed in said base for passage and escape of air from said receptacle. Another embodiment is directed to a receptacle with a bowl, square, star, or cylinder shape. Another embodiment is directed to a holder with a receptacle holding a stick or cone shaped incense product. Another embodiment is directed to a holder wherein the receptacle is integral to the base. Yet another embodiment is directed to a holder with a handle disposed on the receptacle.

To the accomplishment of the above and related objects, this invention may be embodied in the form illustrated in the accompanying drawings, attention being called to the fact, however, that the drawings are illustrative only, and that changes may be made in the specific construction illustrated and described within the scope of the appended claims.

### BRIEF DESCRIPTION OF DRAWINGS

The above and other objects, advantages and features of the present invention will be more readily apparent from the following description, when read in conjunction with the accompanying drawings wherein:

FIG. 1 is a perspective view of an embodiment of the present invention;

FIG. 2 is a perspective view of another embodiment of the present invention with incense accommodated within the receptacle;

FIG. 3 is a front perspective view of another embodiment;

FIG. 4 is a perspective view of an embodiment of the present invention;

FIG. 5 is a perspective view of an embodiment of the receptacle of the present invention as used with stick incense;

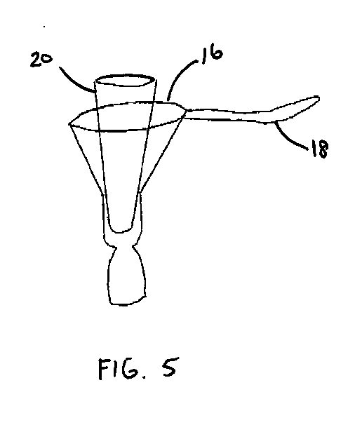

FIG. 6 is a perspective view of an embodiment of the receptacle of the present invention as used with cone incense.

### DETAILED DESCRIPTION

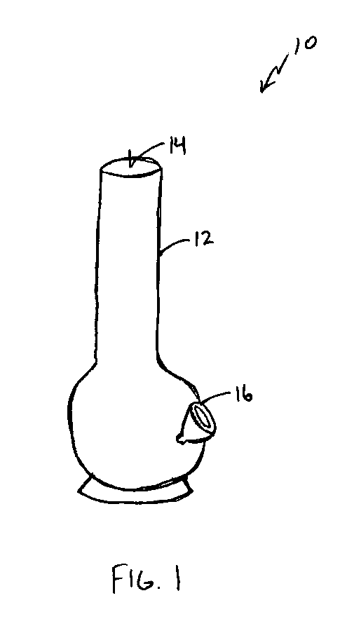

Referring first to FIG. 1, an embodiment of the present invention, generally designated as 10, comprises a vertically disposed base 12 having an inner surface that is configured to be capable of accommodating a candle 14 from the top portion of said base and that is configured to be capable of accommodating a receptacle 16 through an aperture in said base, a removable receptacle disposed in said aperture on a wall of said base that is capable of receiving a fragrant solid or fragrant oil. A handle (not shown) may also be disposed on said receptacle 16.

The structure of candle 14 is inserted into base 12 so that it can then be ignited from its wick located on its top surface. Receptacle 16, which is capable of receiving and holding in place a stick of incense 22, protrudes out from the lower portion of the base and is bowl shaped and hollow. It can be made of plastic, glass or any suitable substance, and it also serves in this embodiment to physically separate the incense 22 from candle 14.

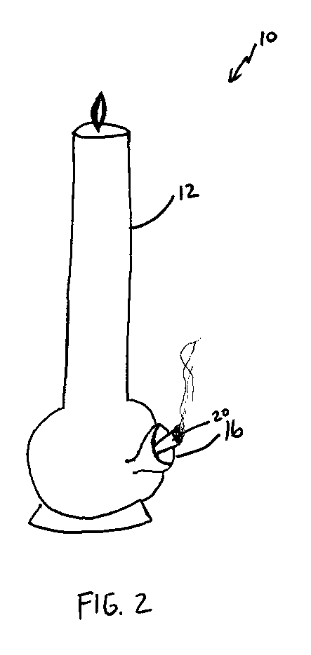

In another embodiment shown in FIG. 2, receptacle 16 is integral to base 12, wherein receptacle 16 is constructed to be a part of base 12. In this embodiment, base 12 also functions as candle 14 and the wick is positioned down the center of the combination holder 10, with the only materials used for holder 10 and receptacle 16 being wick and wax.

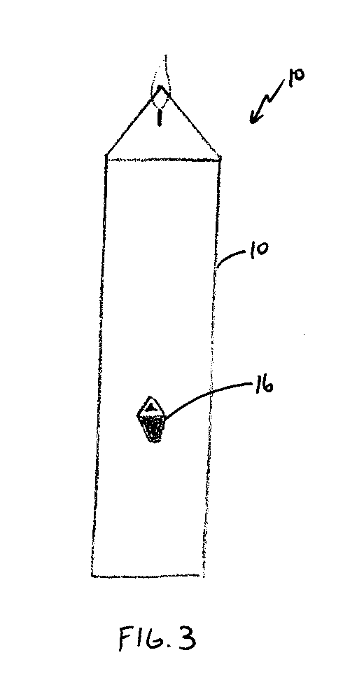

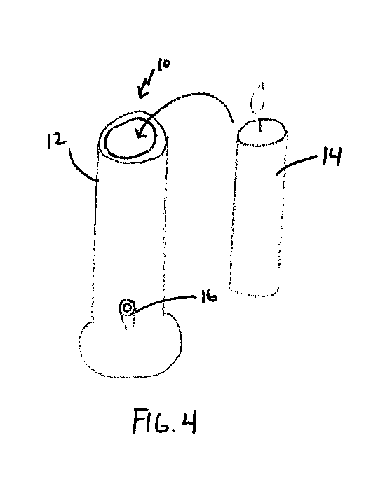

This is in contrast to the first embodiment of FIG. 1, wherein candle 14 is mounted inside the structure of base 12. Candle 14 in both embodiments is ignited at its upper end while the incense stick 22 or cone 20 can be placed in detachable receptacle 16. FIG. 3. shows another embodiment of the holder 10 in the form a triangle. FIG. 4. illustrates how candle 14 can be placed in base 12.

Other embodiments of the present invention can use any commercially available candle, such as a paraffin wax candle, a molded shape candle or a liquid fragrance composition. The size of the wick used will vary depending on the size of the candle. In the instance where a custom mold is used with a wax material for the base portion, it may take about 5 hours for the candle to cool and harden into the desired shape. As the candle cools, objects such as decorative shells and charms may be placed inside of the candle.

In the present embodiment shown in FIG. 2, receptacle 16 holds the stick incense 22 in an upright angle away from candle 14 so that the incense will not melt the candle when the incense 22 is ignited. Cone incense 20 is also prevented from contacting base 12 in the same fashion. Receptacle 16 can be made of glass, ceramic, plastic or any suitable material. When used with cone shaped incense products, the incense 20 should fit snugly into the receptacle. Stick shaped ignitable incense products can be inserted directly into receptacle 16 where incense stick 22 can be securely held in place by receptacle 16. The shape of receptacle 16 can also be varied. For example, it can be in the shape of a bowl, diamond, star, hand or a cone shape and the shape can also facilitate the capture of incense ash as it falls from the fragrant incense into receptacle 16.

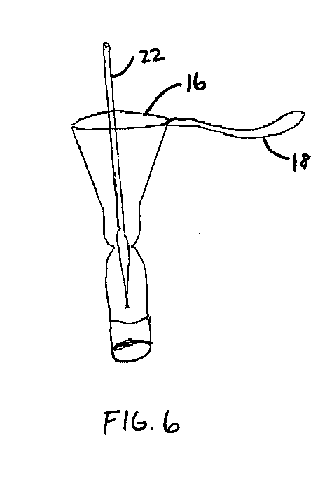

Also in this embodiment, the receptacle can be constructed so that the sides of the receptacle do not come into contact with the incense. FIG. 6 illustrates such an arrangement. In this instance, the stick incense only contacts the bottom of the receptacle which is just large enough to allow the incense stick to be securely held. This allows the incense stick to burn evenly down the length of the stick. In the alternative embodiment shown in FIG. 6, the receptacle is removable with a handle disposed on the receptacle to facilitate removal and placement. The receptacle may be constructed from heat resistant material such as glass or ceramic to prevent heat from transferring from the incense to the base. The receptacle may also be constructed from more than one sections or components to achieve the same result. Where the base is constructed from a wax material, the junction between the base and the receptacle may be reinforced with a sleeve made of copper or other suitable material.

Base 12 can be constructed out of a material such as wax or it can be made of a hard outer substance such as glass or plastic, or any other suitable material. Base 12 can also be refillable with cylinder or other suitably shaped candles, and the candles can also be votive candles or fragrant gel candles. In the case where an ignitable stick incense or an ignitable cone incense is used, there can be an air passageway that is capable of permitting air to flow from the inside to the outside of the base 12.

Base 12 may be constructed with various dimensions such as widths and lengths either wider or narrower and shorter or taller than the embodiments depicted in the figures. Instead of a cylindrical shape, the base may be a square, star, triangular, cone or any other suitable shape. Also, various liquid fragrances disposed in a suitable candle like container may replace and/or be used in addition to the candle.

While the present invention has been illustrated and described by means of specific embodiments and alternatives, it is to be understood that numerous changes and modifications can be made without departing from the spirit and scope of the invention. Therefore, it should be understood that the invention is not to be limited in any way except in accordance with the appended claims and their equivalents.

What is claimed is:

1. A combination candle and incense holder comprising:
a vertically disposed base having an inner surface that is configured to be capable of accommodating a candle from the top portion of said base and that is configured to be capable of accommodating a receptacle through an aperture in said base; and a removable receptacle disposed in said aperture on a wall of said base that is capable of receiving an ignitable fragrant solid or an ignitable fragrant oil.

2. The combination candle and incense holder of claim 1 further comprising a receptacle mounted on the top portion of the housing that is capable of receiving an ignitable fragrant solid or an ignitable fragrant oil.

3. A combination candle and incense holder of claim 1 wherein the base is further comprised of an air well disposed in said base for passage and escape of air from said receptacle.

4. A combination candle and incense holder of claim 1 wherein the receptacle has a bowl-shaped configuration.

5. A combination candle and incense holder of claim 1 wherein the base has a square shape.

6. A combination candle and incense holder of claim 1 wherein the base has a cylindrical shape.

7. A combination candle and incense holder of claim 1 wherein the base has a cone shape.

8. A combination candle and incense holder of claim 1 wherein the base has a star shape.

9. A combination candle and incense holder of claim 1 wherein the incense accommodated in said incense holder has a stick shape.

10. A combination candle and incense holder of claim 1 wherein the incense accommodated in said incense holder has a cone shape.

11. A combination candle and incense holder of claim 1 wherein the receptacle is integral to the base.

12. A combination candle and incense holder of claim 1 further comprising a handle disposed on said receptacle.
